(19) United States
(12) Patent Application Publication
(10) Pub. No.: US 2005/0187607 A1
(43) Pub. Date: Aug. 25, 2005

(54) Drug delivery device

(76) Inventors: Adil Jamal Akhtar, West Bloomfield, MI (US); Syed Abid Mahmood, West Bloomfield, MI (US)

Correspondence Address: DYKEMA GOSSETT PLLC, 10 S. WACKER DR., STE. 2300, CHICAGO, IL, 60606, US

(21) Appl. No.: 10/783,727

(22) Filed: Feb. 20, 2004

(51) Int. Cl. A61F 2/06

(52) U.S. Cl. 623/1.15; 623/1.42; 424/425

(57) ABSTRACT

A medical device for drug delivery is provided which includes a stent structure and a biologically active structure attached to the stent structure. The biologically active structure is comprised of a plurality of layers, including a first layer having a first biologically active compound, such as an anti-proliferative, cytostatic or cytotoxic drug, for delivery at a first target site, such as the vascular tissue; a second layer having a second biologically active compound, such as a growth factor, for delivery into the arterial lumen and capable of promoting engraftment and differentiation of hematopoietic stem cells and/or endothelial progenitor cells at a second site of myocardial injury; and a third, middle layer which is substantially or selectively impermeable to both the first and second biologically active compounds.

### BACKGROUND OF THE INVENTION

1. Field of the Invention

The present invention relates generally to a medical device, and more specifically to a stent having a biologically active layered structure which releases a first biologically active compound and a second biologically active compound, wherein the first and second biologically active compounds may be insulated from each other by an impermeable layer and further may have mutually antagonistic biological activities.

2. Discussion of Related Art

Ischemia is a condition characterized by a lack of oxygen supply in tissues of organs due to inadequate perfusion. Ischemic cardiomyopathy occurs when the arteries that supply blood and oxygen to the heart are blocked, leading to myocardial cell damage and loss of myocardial function. Ischemic cardiomyopathy is a frequent cause of congestive heart failure and remains a leading cause of morbidity and mortality in the United States and worldwide. Myocardial infarction (MI) is caused by a sudden and sustained lack of blood flow to an area of the heart commonly caused by obstruction of a coronary artery. Without adequate blood supply, the tissue becomes ischemic, leading to the death of myocytes and vascular structures. Treatment includes an attempt to restore the flow of blood to the affected area by a procedure referred to as percutaneous transluminal coronary angioplasty (PTCA).

In PTCA, a catheter with a deflated balloon is inserted and advanced to the narrow part of the selected artery. The balloon is then inflated, thereby enlarging the inner diameter of the blood vessel. The balloon is deflated and the catheter removed. PTCA may also involve the placement of a stent. A stent is an expandable hollow tube used to maintain an open passageway in an artery after PTCA. The stent is initially in a collapsed, unexpanded state with a small diameter before and during its advancement to the area of occlusion or narrowing in the artery. The stent is expanded, either through self-expansion or with the aid of an expanding balloon catheter placed within the stent. The stent is expanded in place and effectively forms a scaffold holding the artery open.

Restenosis (renarrowing or reocclusion) is a known problem with stents, and is due primarily to neointimal hyperplasia. Stent implantation typically creates arterial injury provoking an inflammatory response and smooth muscle cell (SMC) migration and proliferation. The prior art stent technology has focused on drug-eluting stents, which are coated with drugs which, when released, help to keep the blood vessel from renarrowing or reoccluding. These drugs may be coated directly on the stent or may be incorporated into a polymer coating applied to the stent. These drugs are generally anti-proliferative and anti-inflammatory, and further may be cytostatic or cytotoxic in order to prevent restenosis.

Prior art stent technology has thus far remained focused on preventing such restenosis of the stent. While the delivery of anti-proliferative drugs to the vascular tissue at the site of the arterial occlusion may reduce the potential for such restenosis, there remains the additional concern for recovery of myocardial function. A need remains, therefore, for a localized, in vivo delivery system of one or more biologically active compounds, such as growth factors, that will help to repair, generate and/or regenerate myocardial tissue. Such a delivery system should avoid interfering with any delivery of anti-proliferative drugs which prevent restenosis, and further should avoid inducing side effects generally associated with systemic administration of compounds such as growth factors.

### SUMMARY OF THE INVENTION

The present invention provides a medical device for drug delivery. The medical device includes a stent structure having an outer surface and an inner surface that defines a lumen. A biologically active structure is attached to the stent structure and comprises a plurality of layers. The first layer of the biologically active structure incorporates a first biologically active compound having a first biological activity. The second layer of the biologically active structure incorporates a second biologically active compound having a second biological activity. A third layer of the biologically active structure is located between the first and second layers and is substantially impermeable to both the first biologically active compound and to the second biologically active compound. In selected embodiments, the first biological activity and the second biological activity have mutually antagonistic or otherwise opposing activities. The third layer may also be selectively impermeable to the first biologically active compound and to the second biologically active compound.

The various embodiments of the medical device for drug delivery in accordance with the present invention are advantageous compared to existing stents or other drug delivery devices for sites of arterial occlusion. The inventive drug delivery device enables the delivery of two drugs to two different target sites with potentially different and opposing or antagonistic actions. The various embodiments of the invention are capable of providing cytotoxic and cytostatic drugs to the vascular tissue surrounding the implanted stent, in order to reduce inflammation and SMC proliferation, and thereby reduce restenosis. The various embodiments of the invention are also capable of providing growth factors locally to the arterial lumen to facilitate endothelialization, and further to the bloodstream in order to promote the viability, engraftment, and differentiation of endothelial progenitor cells, hematopoietic stem cells and/or other progenitor or stem cells downstream from the arterial occlusion, thereby facilitating myocardial tissue regeneration.

In addition to delivering potentially antagonistic drugs, the various embodiments of the invention provide an impermeable layer within the biologically active structure to insulate the two biologically active compounds, which may have both potentially opposing biological activities and also different target sites, such that each biologically active compound may be effective without interference or inhibition from the other biologically active compound. Additionally, the localized in vivo method of delivery of biologically active compounds, such as growth factors, is capable of preventing side effects associated with systemic administration of growth factors.

These and other features and objects of this invention will become apparent to one skilled in the art from the following detailed description and the accompanying drawings illustrating features of this invention by way of example.

### BRIEF DESCRIPTION OF THE DRAWINGS

FIG. 1 illustrates a perspective view of an exemplary first embodiment of the present invention with the biologically active structure attached to an outer surface of a stent structure.

FIG. 2 illustrates a perspective view of the exemplary first embodiment of the present invention showing exposed layers of the biologically active structure attached to the outer surface of the stent structure.

FIG. 3 illustrates a radial, cross-sectional view of the exemplary first embodiment of the present invention with the biologically active structure attached to the outer surface of the stent structure.

FIG. 4 illustrates a perspective view of an exemplary second embodiment of the present invention showing exposed layers of the biologically active structure attached to an inner surface of a stent structure.

FIG. 5 illustrates a radial cross-sectional view of the exemplary second embodiment of the present invention with the biologically active structure attached to the inner surface of the stent structure.

FIG. 6 illustrates a perspective view of an exemplary third embodiment of the present invention showing exposed layers of the biologically active structure interleaved between and among inner and outer surfaces of a stent structure.

FIG. 7 illustrates a perspective view of an exemplary fourth embodiment of the present invention showing exposed layers of the biologically active structure attached to both inner and outer surfaces of a stent structure.

### DETAILED DESCRIPTION OF EMBODIMENTS OF THE INVENTION

The present invention provides a drug delivery system capable of administering one or more biologically active compounds with potentially opposing, antagonistic, synergistic, or unrelated biological activities to two or more separate target sites. Such target sites include, for example, a first, arterial region local to an implanted stent, where cytotoxic or cytostatic medications are useful to prevent restenosis, and a second region downstream from the first, such as the cardiac region adversely impacted by a MI, where growth factors (having a biological activity antagonistic to that of the cytotoxic or cytostatic medications) are useful to aid tissue regeneration. Additionally, depending upon their potentially antagonistic biological activities, in accordance with the invention, these biologically active compounds are maintained substantially insulated from each other. The present invention further provides for localized, in vivo delivery of the biological compounds in order to encourage endothelial and myocardial tissue regeneration, without substantial systemic side effects.

As discussed above, in accordance with the present invention, the delivery of cytotoxic or cytostatic drugs to the vascular tissue surrounding an implanted stent may reduce restenosis, and the concomitant delivery of growth factors also may facilitate the endothelialization of the stent, thereby having the further capability of preventing stent thrombosis (which is associated with delayed endothelialization). In addition, the present invention avoids a problem of prior art stents, in which the population and activity of hematopoietic (or hematopoetic) stem cell, endothelial progenitor cells and/or other progenitor or stem cells at the site of myocardial infarction or chronic critical ischemia, as well as at the site of arterial narrowing (PTCA or stent site), may be damaged or restricted if exposed to the cytotoxic or cytostatic drugs, which are released from the drug eluting stents. This damage in the prior art from the cytotoxic or cytostatic drugs may impair the recovery of myocardial function and delay the process of endothelialization of the stent.

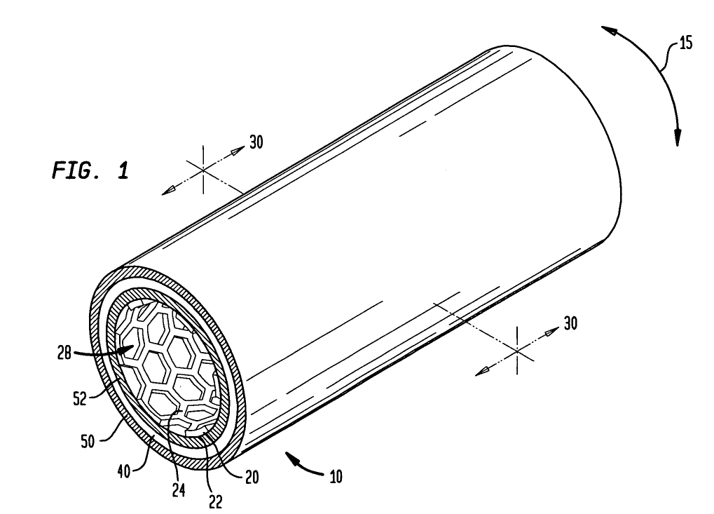

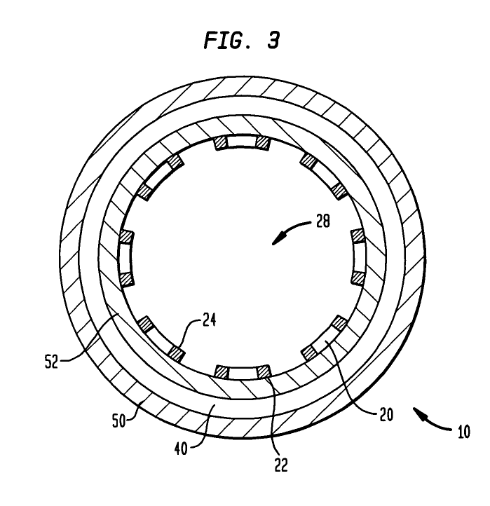

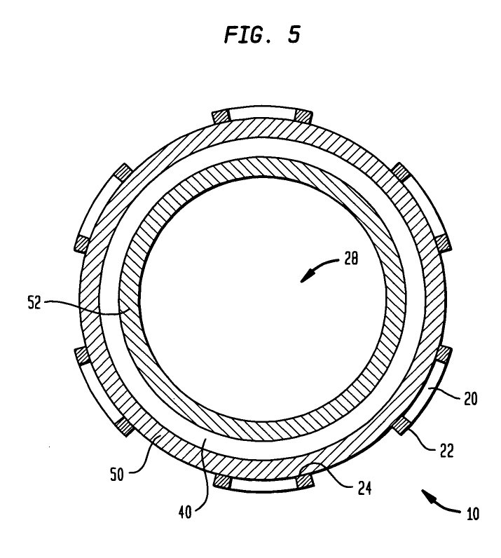

Referring now to the drawings wherein like reference numerals are used to identify identical components in the various views, FIGS. 1-7 illustrate a medical device 10 of the present invention and its various components, including a stent structure 20 and a biologically active structure 60. The biologically active structure 60 comprises a plurality of layers, a first (generally polymer) layer 50 of the plurality of layers having a first biologically active compound, a second (generally polymer) layer 52 of the plurality of layers having a second biologically active compound, and a third, impermeable layer 40, of the plurality of layers, located between the first and second (polymer) layers 50, 52 and insulating or separating the first and second biologically active compounds.

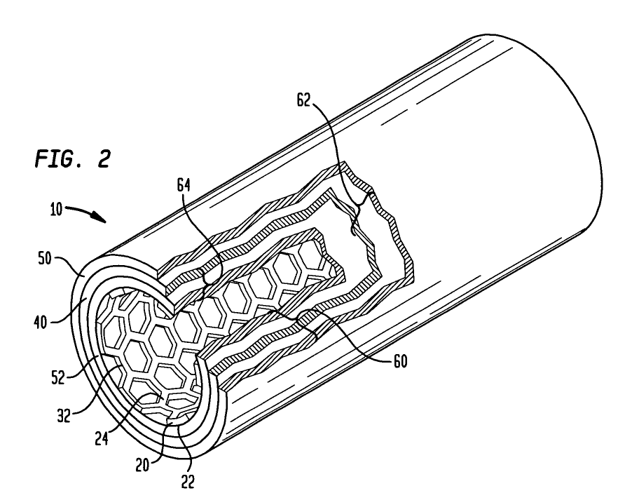

In FIGS. 2, 4, and 7, various layers of the medical device 10 are exposed to illustrate the present invention in greater detail, it being understood that in actual use and fabrication, these layers are not exposed, and instead each layer extends in the longitudinal and radial directions substantially for the entire (or partial (and variable)) length or circumference of the stent structure 20. Depending upon the selected embodiment, alternatively, each layer may not necessarily span the entire length (along axis 30) or circumference (illustrated as radial or circumferential axis 15) of the stent, but may span partial lengths (or circumferential portions), which also may be variable, may be located only at the proximal and distal ends of the stent, for example, or may extend in numerous other variations which will be apparent to those of skill in the art. FIG. 1 illustrates a perspective view of such an exemplary, complete medical device 10 for drug delivery in accordance with one embodiment of the present invention, and illustrates the biologically active structure and its composite layers 40, 50, and 52 extending for both the complete length and circumference of the stent structure 20.

The medical device 10 may also be generally referred to as a “stent”. When the term “stent structure” is used herein, however, the term refers not to the complete medical device 10 of the present invention, but merely to that portion of the medical device labeled as element 20. The skilled artisan will recognize that there are a multiplicity of different and equivalent stent structures, having different configurations and structural elements that may be utilized in accordance with, and are within the scope of, the present invention, such as those shown and described in U.S. Pat. Nos. 6,120,536, 6,656,156 and Frederick G. P. Welt, MS, MD, Piotr S. Sobiesczyk, MD, Coronary Artery Stents: Design And Biologic Considerations, Cardiology Special Edition, Vol. 9. Several exemplary stent structures 20 will be illustrated in the accompanying drawings, it being understood that the exemplary stent structure 20 may have innumerable configurations and structural elements, such as open or closed structures, ring structures, self-expanding structures, modular structures, and so on.

FIGS. 1-7 illustrate a stent structure 20, in an expanded state. The stent structure 20 has an outer surface 22 and an inner surface 24 defining a lumen 28. The stent structure is generally tubular and comprises a first end and a second end with an intermediate section there between. The stent structure has a longitudinal axis (illustrated as axis 30) and comprises a plurality of segments or struts 32. In the exemplary embodiments, the stent structure 20 is biocompatible and non-thrombogenic, and further may be bioresorbable or corrosion-resistant. Additionally, the stent structure 20 may have radiographic visibility and be least antigenic.

The stent structure 20 is usually formed having a comparatively small diameter, i.e., in a collapsed or unexpanded state. When inserted at its target, arterial site, the stent structure 20 is expanded circumferentially, either through self-expansion or with the aid of an expanding balloon catheter placed within the stent structure. Generally, balloon expandable stent structures are comprised of stainless steel, while self-expanding stent structures are comprised of nitinol, a nickel-titanium alloy. Stent structures may also be made of a cobalt-chromium alloy, magnesium alloy, gold, platinum, inconel, iridium, silver, tungsten, carbon, carbon fiber, silicone, cellulose acetate, cellulose nitrate, or biodegradable or bioresorbable polymers, and a wide variety of numerous other polymers, such as polyurethane, polyester, including many of the polymers discussed below with reference to layers 50, 52. It is expected that newer stent structures will be comprised of bioresorbable materials. When inserted at the target location, the stent structure is maintained in an expanded configuration that is circumferentially rigid.

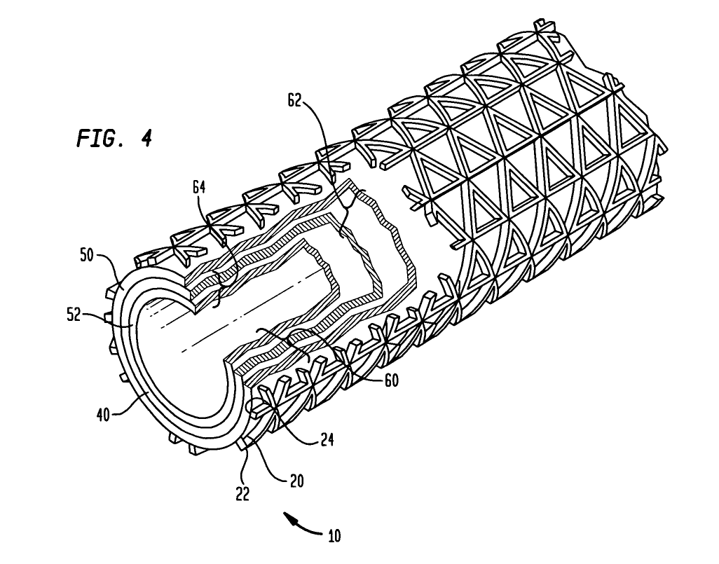

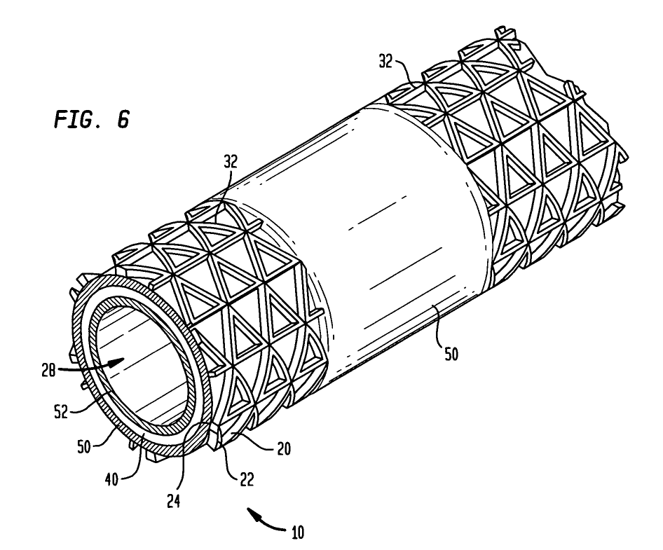

The medical device 10 further includes a biologically active structure 60. Biologically active structure 60 comprises a plurality of layers, including a first (polymer) layer 50, a second (polymer) layer 52, and a third, impermeable layer 40. Biologically active structure 60 may be attached to the outer surface of stent structure 20 as shown in FIG. 2. In a second exemplary embodiment, biologically active structure 60 may be attached to the inner surface of stent structure 20 as shown in FIG. 4. In a third exemplary embodiment, biologically active structure 60 may be interleaved between the inner surface of stent structure 20 and the outer surface of stent structure 20, such as on alternate struts 32 (or rings) or otherwise between or among the struts 32 (or rings); such interleaving is illustrated in an exemplary, albeit simplified form in FIG. 6, with interleaving between groups of several struts 32, it being understood that such interleaving may occur in a wide variety of equivalent ways within the scope of the invention, such as interleaving between and among individual struts 32, cells, rings, or spaces. In a fourth exemplary embodiment, biologically active structure 60 is effectively divided into two portions, 62 and 64, with the impermeable layer 40 attached to both the inner and outer surfaces of stent structure 20 and effectively having the stent structure 20 embedded in the biologically active structure 60.

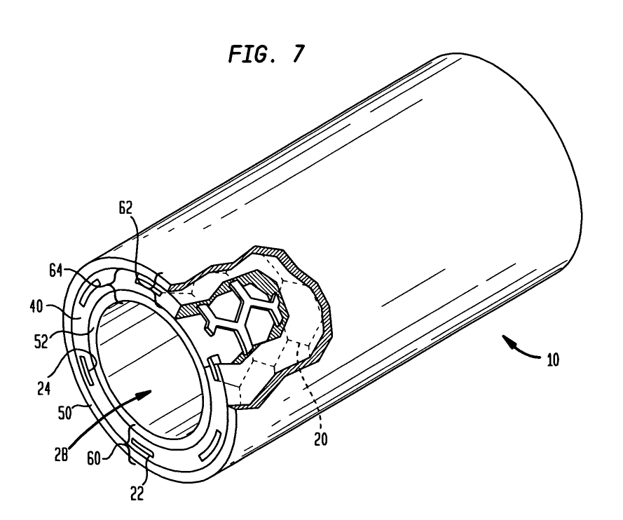

More specifically, in the fourth exemplary embodiment, biologically active structures 62 and 64 may be utilized as shown in FIG. 7. Biologically active structure 62 includes an impermeable layer 40 and polymer layer 50. Biologically active structure 62 is attached to the outer surface of stent structure 20. Biologically active structure 64 includes an impermeable layer 40 and polymer layer 52. Biologically active structure 64 is attached to the inner surface of stent structure 20. It should be noted that the impermeable layer 40 may be applied to both the outer surface and the inner (luminal) surface of the stent structure 20 as one integral layer, as illustrated in FIG. 7, effectively embedding the stent structure 20 in the impermeable layer 40 of the biologically active structure 60. Alternatively, biologically active structures 62 and 64 may each utilize separate or otherwise non-integral impermeable layers 40.

While not separately illustrated, in a fifth exemplary embodiment of the present invention, only biologically active structure 62 is attached to either the outer surface or inner surface of stent structure 20. Similarly, in a sixth exemplary embodiment, also not separately illustrated, only biologically active structure 64 is attached to either the outer surface or inner surface of stent structure 20. These embodiments provide for the delivery of one or more drugs to a single target site while preventing the delivery of these drugs to any unwanted locations, such as providing for a first biological compound having cytotoxic activity to be limited to delivery to the portion of an arterial wall abutting the medical device 10 and substantially insulated from elution downstream, or providing for a second biological compound having cell stimulating activity limited to downstream delivery and substantially insulated from the local arterial wall abutting the medical device 10. Numerous other variations will be apparent and are also within the scope of the present invention, such as providing in various combinations for one layer to be on the outer or inner surface of the stent structure 20, the impermeable layer to be on the outer or inner surface (or both) of the stent structure 20, and the second layer to be on the outer or inner surface of the stent structure 20, i.e., interspersing the biologically active structure 60 with the stent structure 20 or, equivalently, incorporating the stent structure 20 within or between any of the various layers (40, 50, 52) of the biologically active structure 60. (As used herein, “drug” or “biologically active compound” are interpreted broadly, to mean and include any compound which has a selected or desired pharmacologic effect.)

In a seventh exemplary embodiment of the present invention, the biologically active structure comprises an impermeable layer 40 separating one or more biologically active compounds on either surface (as layers 50, 52) of the impermeable layer. These biologically active compounds may be incorporated in a plurality of layers, or may be incorporated in a mosaic, mixed or interspersed pattern on either surface of the impermeable layer, or any combination thereof In the mosaic pattern, different biologically active compounds may be located in a single layer adjacent to each other in various patterns, rather than in different layers covering the same surface of the impermeable layer. These variations of both layering and mixtures of a plurality of biologically active compounds, all within the scope of the invention and applicable to any of the embodiments, allow for elution or release of any of a plurality of biologically active compounds, selectively, at variable times, for variable durations, having variable dosage levels, and having variable phases, which may be predeterminable or programmable, as discussed in greater detail below.

In various selected embodiments, the third, impermeable layer 40 of biologically active structure 60 is generally a middle layer located between a first (polymer) layer 50 and a second (polymer) layer 52, as shown in FIGS. 1-6. Impermeable layer 40 also may be located next to only one (polymer) layer 50, 52, depending on whether it is formed as two separate layers, rather than as one integral layer as shown in FIG. 7, or if only one of the biologically active structures 62 or 64 is included in the medical device 10. Impermeable layer 40 may be made of a biocompatible polymer or other material, and may be formed as part of the first and second layers 50, 52, or as a separate component, such as a membrane, or as a separate polymer layer. While third layer 40 is referred to as “impermeable”, it should be understood that such impermeability, as used herein, is relative or comparative, as a matter of pharmacological (or biological) sufficiency, and is not required to be absolutely impermeable or 100% impermeable. More specifically, the degree of impermeability for a selected embodiment is based upon both the comparative or relative pharmacological or biological needs for the selected purposes and effects of the first or second biologically active compounds and the treatment objectives, particularly in light of various factors. Such factors include, without limitation, the rate of loss of effectiveness or other degradation or metabolism of these compounds and their active metabolites; any degree of tolerable or acceptable leakage or elution without significantly adverse effects; the rate of bioresorption of the layer; and further in light of any requisite or advisable treatment durations, and other relevant factors. For example, for selected biologically active compounds, requisite impermeability may be comparatively short term, measured in hours or days, while in other circumstances impermeability for longer durations is advisable, such as for several months. Also for example, depending upon the selected embodiment, some degree of permeability may be tolerable, depending upon whether such leakage or elution causes adverse effects, such as inhibiting the effectiveness of other biologically active compounds of other layers. In other circumstances, the biological sufficiency of the degree of impermeability may be empirically determined. The third layer may also be selectively impermeable to the first biologically active compound and to the second biologically active compound, such as based on their polarities, molecular size, concentrations, or lipid permeability (e.g., lipophilic or lipophobic compounds). The impermeability of the third layer 40 may also be programmable or predeterminable, for example, through selection of composite materials, through the application of electrical or ultrasound energy, through selection of composite material cure rates, etc. Such selective or programmable impermeability generally will also be based on pharmalogical or biological sufficiency criteria, for a selected application or treatment objective, as discussed above. (In addition, depending upon the configuration of the biologically active structure 60 and its composite layers in any selected embodiment, some degree of non-impermeability or leakage may be expected from the structure 60, such as from any exposed ends or edges of the first and second layers which may not be fully covered by layer 40 in a given embodiment.)

The impermeable layer 40 may further be impervious, bioresorbable or biodegradable, non-allergenic, and non-thrombotic. The term “biocompatible” when used in relation to polymers is recognized in the art. Biocompatible polymers include polymers that are neither themselves toxic to the body, nor degrade (if the polymer degrades) at a rate that produces monomeric or oligomeric subunits or other byproducts that are toxic or are produced at toxic concentrations in the host. Impermeable layer 40 may be made of an elastomeric material so that it may expand correspondingly with the expansion of the stent structure 20 during a PTCA procedure. The material should be sufficiently elastomeric to allow for expansion by up to several times or more of its unexpanded diameter.

Examples of materials used for the impermeable layer 40 may include, but are not limited to, polymeric materials such as ethylene vinyl acetate, latexes, urethanes (such as polycarbonate urethane), polysiloxanes, styrene-ethylene/butylene-styrene block copolymers, silicone rubber, Silastic™, aliphatic polyesters, and mixtures and copolymers thereof. Other materials which may be utilized in layers 50, 52, as discussed below, may also be utilized in impermeable layer 40, providing the material(s) meet the impermeable (or biologically sufficient insulation) criterion for impermeable layer 40. In an exemplary embodiment of the present invention, the impermeable layer 40 is one layer in a multi-layer polymer. The impermeable layer 40 may be applied to (polymer) layers 50, 52 or the stent structure 20 itself (as shown in FIG. 7), by spray or dip-coating processes, for example. The polymer making up the impermeable layer 40 may be sprayed onto polymer layer 50, 52 or stent structure 20 and allowed to dry. In other embodiments, the first and second layers 50, 52 may be applied to the impermeable layer 40. In another exemplary embodiment, during a spraying process, the polymer making up impermeable layer 40 may be electrically charged to one polarity and the polymer layer 50 or 52 may be electrically charged to the opposite polarity. In this manner the impermeable layer 40 and polymer layer 50 or 52 will be attracted to one another. With this type of spray process, waste may be reduced and more control over the thickness of the impermeable layer 40 may be achieved.

In another exemplary embodiment, the impermeable layer 40 may be a separate membrane. Examples of material to be used to form the membrane include polytetrafluoroethylene (Teflon) and membranes formed from the various polymers described below. The membrane is of minimal thickness to minimize the profile of the medical device 10.

The biologically active structure 60 of the medical device 10 further includes layers 50, 52. While in the exemplary embodiments layers 50, 52 are each generally one or more polymeric layers or sublayers incorporating one or more biologically active compounds therein (and may be referred to as a polymeric matrix, as discussed below), in other embodiments the layers 50, 52 each may be comprised of other materials which may or may not be polymeric, or may be comprised of one or more biologically active compounds which are applied to the impermeable layer 40, for example, as coatings, potentially with or without other or additional polymeric or nonpolymeric materials. As a consequence, reference herein to polymer layers 50, 52 shall be understood to mean and include, more generally, corresponding layers 50, 52, and vice-versa, and each such layer 50, 52 may also include or be comprised of a plurality of sublayers or other form of polymeric matrix, as discussed below.

When medical device 10 is inserted, in a PTCA process for example, polymer layer 50 may abut the vascular tissue when biologically active structure 60 is attached to the outer surface of stent structure 20, or polymer layer 50 may be exposed to the vascular tissue through openings or fenestrations in stent structure 20 if biologically active structure 60 is attached to the inner surface of stent structure 20. When medical device 10 is inserted, polymer layer 52 may be exposed to the arterial lumen through openings or fenestrations in the stent structure 20 if biologically active structure 60 is attached to the outer surface of stent structure 20, or polymer layer 52 may be more directly exposed to the lumen if biologically active structure 60 is attached to the inner surface of stent structure 20. As mentioned above, given the other equivalent variations of the relationship between the biologically active structure 60 and the stent structure 20, there are innumerable other variations, all within the scope of the present invention, such that layer 50 is primarily or exclusively exposed to or abutting the vascular tissue, and layer 52 is primarily or exclusively exposed to the arterial lumen.

Polymer layers 50, 52 may be made of non-inflammatory, non-thrombogenic, biocompatible substances. Polymer layers 50, 52 also may have elastomeric properties so that they may expand correspondingly with the expansion of the stent structure 20 and further be protected from surface integrity changes such as cracking or peeling. Polymer layers 50, 52 may further be biodegradable. Polymer layers 50, 52 may provide programmable or predeterminable drug elution kinetics, and generally should also not interfere with the first or second biologically active compounds.

Any number of polymers may be utilized as polymer layers 50, 52. Although in FIGS. 1-7, polymer layers 50, 52 are illustrated as single layers, each polymer layer 50, 52 made be comprised of a plurality of sublayers incorporating one or more biologically active compounds, and may be referred to collectively as a polymeric matrix. A biologically active compound may be incorporated in a first sublayer, and a second sublayer may serve as a barrier to diffusion in order to control the elution of the biologically active compound from the first sublayer, for example. In an exemplary embodiment, a polymeric matrix forming either layer 50, 52 (or both), comprising a polymer and an incorporated biologically active compound, may be formed as two sublayers. The first sublayer comprises a solution of ethylene-co-vinylacetate and polybutylmethacrylate. The first or second biologically active compound, respectively, is incorporated in this layer. The second outer sublayer comprises only polybutylmethacrylate and acts as a diffusion barrier to prevent the respective first or second biologically active compound from eluting too quickly into the surrounding tissue. In the exemplary embodiments, the total thickness of such a polymeric matrix ranges from about 1 micron to 20 microns or greater.

The layers 50, 52 (including sublayers or polymeric matrices) may be applied to the impermeable layer 40 in several ways, and vice-versa. For example, the layers 50, 52 (or polymeric matrix) may be sprayed onto the impermeable layer 40, or the impermeable layer 40 may be dip-coated with the layers 50, 52 (including any sublayers or polymeric matrices). These various coatings may be applied by dipping or spraying using evaporative solvent materials of relatively high vapor pressure. In an exemplary embodiment, the layers 50, 52 (or the corresponding sublayers or polymeric matrices) are sprayed onto the impermeable layer 40 and then allowed to dry. In another exemplary embodiment, in the spraying process, each layer 50, 52 (including sublayers or polymeric matrix) may be electrically charged to one polarity and the impermeable layer electrically charged to the opposite polarity. In this manner, the polymeric matrices (sublayers or layers 50, 52) and impermeable layer will be attracted to one another. In using this type of spraying process, waste may be reduced and more control over the thickness of the polymeric matrix, sublayers, layers 50, 52, or other layered coatings may be achieved.

In another exemplary embodiment, to form the polymer layers 50, 52 (including formed as sublayers or as polymeric matrices), the biologically active compound may be incorporated into a film-forming polyfluoro copolymer comprising an amount of a first moiety selected from the group consisting of polymerized vinylidenefluoride and polymerized tetrafluoroethylene, and an amount of a second moiety other than the first moiety and which is copolymerized with the first moiety, thereby producing the polyfluoro copolymer, the second moiety being capable of providing toughness or elastomeric properties to the polyfluoro copolymer, wherein the relative amounts of the first moiety and the second moiety are effective to provide each coating forming one of the layers 50, 52, respective sublayers or polymeric matrix. The polyfluoro copolymer coatings may be applied in one or more coating steps. It may be highly advantageous to use a diluted first coating solution comprising a polyfluoro copolymer as a primer to promote adhesion of a subsequent polyfluoro copolymer coating layer that may include the first biologically active compound.

Each of the polymer layers 50, 52 (including sublayers or polymeric matrices) may also comprise a “hydrogel.” A hydrogel can be a synthetic polymer, such as polymalic acid, polyamino acids, polyacrylic acids, polyethylene glycol, polyvinylpyrrolidone, polyvinyl alcohols, and hydrophilic polyurethanes. Hydrogels can include albumin, collagen, gelatin, starch, cellulose, dextran, polymalic acid, polyamino acids and their co-polymers or lightly cross-linked forms. Other possible materials forming the layers 50, 52 (sublayers or polymeric matrices) are polysaccharides and their derivatives, sodium alginate, karaya gum, gelatin, guar gum, agar, align, carrageenans, pectin, locust bean gums, xanthan, starch-based gums, hydroxyalkyl and ethyl ethers of cellulose, sodium carboxymethylcellulose.

Polymer layers 50, 52, respective sublayers or polymeric matrices, also may be made of one or more of the following: poly(amides) such as poly(amino acids) and poly(peptides); poly(esters) such as poly(lactic acid), poly(glycolic acid), poly(lactic-co-glycolic acid), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art, copolymers and mixtures thereof).

Representative synthetic polymers for use in polymer layers 50, 52, sublayers, or in polymeric matrices, further include: poly(ethers) such as poly(ethylene oxide), poly(ethylene glycol), and poly(tetramethylene oxide); vinyl polymers such as poly(acrylates) and poly(methacrylates) such as methyl, ethyl, other alkyl, hydroxyethyl methacrylate, acrylic and methacrylic acids, and others such as poly(vinyl alcohol), poly (vinyl pyrolidone), and poly(vinyl acetate); poly(urethanes); cellulose and its derivatives such as alkyl, hydroxyalkyl, ethers, esters, nitrocellulose, and various cellulose acetates; poly(siloxanes); and any chemical derivatives thereof (substitutions, additions of chemical groups, for example, alkyl, alkylene, hydroxylations, oxidations, and other modifications routinely made by those skilled in the art), copolymers and mixtures thereof.

Additional examples of polymers for use in layers 50, 52 (including sublayers or polymeric matrices) include plasticized nylon, plasticized soft nylon, natural rubber, silicone, silicone rubbers of medical grade, ethylene-propylene rubber, silicone-carbonate copolymers, poly(olefins), poly(vinyl-olefins), poly(styrene), poly(halo-olefins), poly(isobutylene), polylactide, polylactide-co-glycolide, polydioxanone, thermoplastic elastomers, thermpoplastics, expanded PTFE, poly(vinyl-chloride), poly(isoprene), poly(isobutylene), poly(butadiene), or a mixture thereof.

In an exemplary embodiment, as described previously, a first biologically active compound can be incorporated into polymer layer 50 or any sublayers, thereby creating a polymeric matrix. It is understood that the first biologically active compound may alternatively be coated directly onto the outer periphery 22 of the stent structure 20 or it may be coated directly onto a first surface of impermeable layer 40. The first biologically active compound may be present as a liquid, a finely divided solid, or any other appropriate physical form. Finely divided means any type or size of included material from dissolved molecules through suspensions, colloids, and particulate mixtures. Optionally, additives such as diluents, carriers, excipients, stabilizers or the like may be added to the polymeric matrix, in addition to the first or second biologically active compound. Correspondingly, using similar means, a second biologically active compound may be incorporated into polymer layer 52 or any sublayers.

In the coating process, a solution of the biologically active compound is generally mixed with a solution of the polymer, and then the mixture is applied to the surface of the impermeable layer 40 by the methods mentioned earlier such as dip coating, spray coating, brush coating, or combinations thereof, and any solvent is allowed to evaporate. The biologically active compound may not be dissolved, but may be mixed or suspended in the solvent. Alternatively to applying a mixture of the biologically active compound (solution, suspension or solid particles) with the polymer solution, the polymer solution and biologically active compound solution may be applied separately.

The term “incorporated” is recognized in the art when used in reference to a therapeutic agent, or other biologically active compound, and a polymeric composition, such as a composition of the present invention. In certain embodiments, this term includes incorporating, formulating, or otherwise including such biologically active compound into a composition or mixture that allows for release, such as sustained release, of such biologically active compound in the desired application. The term contemplates any manner by which a biologically active compound is incorporated into a polymeric matrix, including for example: attached to a monomer of such polymer (by covalent, ionic, or other binding interaction), physical admixture, enveloping the biologically active compound in a coating layer of polymer, and having such monomer be part of the polymerization to give a polymeric formulation, distributed throughout the polymeric matrix, appended to the surface of the polymeric matrix (by covalent or other binding interactions), encapsulated inside the polymeric matrix, etc.

The first biologically active compound, incorporated in layer 50, is generally anti-proliferative and anti-inflammatory. Furthermore, it is cytostatic or cytotoxic. For example, the first biologically active compound may comprise one or more of the following anti-proliferative, anti-inflammatory, anti-coagulant, cytotoxic or cytostatic agents: rapamycin, heparin, anti-thrombin compounds, prostaglandin inhibitors, platelet inhibitors, taxol and taxol derivatives, tacrolimus and tachrolimus-containing compounds, cytochalasin, paclitaxel, dexamethasone, steroids, methotrexate, etc. In selected embodiments, the one or more first biologically active compounds used in the present invention are selected from a number of therapeutic agents depending on the desired application. For example, these therapeutic agents include anti-inflammatory agents such as dexamethasone, prednisolone, corticosterone, budesonide, estrogen, sulfasalazine, mesalamine, and analogues thereof; antineoplastic, antiproliferative, and/or antimiotic agents such as paclitaxel, 5-fluorouracil, cisplatin, vinblastine, vincristine, epothilones, endostatin, angiostatin, tyrosine kinase inhibitors, and analogues thereof; anesthetic agents such as lidocaine, bupivacaine, ropivacaine, and analogues thereof; and anti-coagulants, for example. (Also included, for either the first or second biologically active compound, are nucleic acid compounds such as antisense oligonucleotides, ribozymes, and genes carried by viral vectors (retro, adeno, adenoassociated, lenti, ebola, herpes simplex, etc.) and non viral systems (plasmid, cationic lipid materials, compacting agents, etc.)) When inserted, the first biologically active compound is delivered to the vascular tissue surrounding the outer surface 22 of the stent structure 20, and is capable of preventing or reducing neointimal hyperplasia (which is the predominant mechanism for restenosis of coronary arteries after successful angioplasty and stenting).

When the medical device is inserted, the first (or second) biologically active compound elutes from the polymeric matrix over time and enters the surrounding tissue (or plasma), typically by diffusion. The delivery of the first (or second) biologically active compound may be immediate or delayed. The rate of diffusion can be controlled by altering the strength of the polymer in the respective polymer layer 50, 52. The rate of diffusion can also be controlled by altering the ratio of the polymer to the first (or second) biologically active compound in the polymer layer 50, 52. A higher ratio of polymer to first (or second) biologically active compound will result in a slower release. The thickness of any outer sublayer used in forming a polymeric matrix also may determine the rate at which the first biologically active compound elutes from the polymeric matrix. A thicker outer sublayer will result in a slower rate of release of the first (or second) biologically active compound. Essentially, the first (or second) biologically active compound elutes from the polymeric matrix generally by diffusion through the polymer molecules, although other forms of release of the first biologically active compound are within the scope of the present invention. In selected embodiments, the release of the first (or second) biologically active compound may occur in phases, with a first release phase of the first biologically active compound occurring for approximately a 24-48 hour period, and a second, slower release phase lasting from approximately 2-4 weeks and up to 90 days, for example.

In an exemplary embodiment, a second biologically active compound can be incorporated into a polymer to form layer 52 or any sublayers, also thereby creating a polymeric matrix. It is understood that alternatively the second biologically active compound may be coated directly onto the inner surface 22 of the stent structure 20 or it may be coated directly onto a second surface of impermeable layer 40. The functions, application, and control over elution kinetics of the second biologically active compound of polymer layer 52 may be completed in the same manner as the application of polymer layer 50 described previously.

In selected embodiments, the second biologically active compound comprises one or more growth factors. Without limitation, the growth factor may be one or more of the following exemplary growth factors: granulocyte colony-stimulating factor (G-CSF or neupogen^{™}), granulocyte-macrophage colony-stimulating factor (GM-CSF), CSF-1, G-CSF Ser. sup. 17, M-CSF, c-mpl ligand (MGDF or TPO), erythropoietin (EPO), stem cell factor (SCF), interleukins 1-16 (IL-1, IL-2, IL-3, IL-4, IL-5, IL-6, IL-7, IL-8, IL-9, IL-10, IL-11, IL-12, IL-13, IL-14, IL-15, IL-16), flt3 ligand, human growth hormone, B-cell growth factor, B-cell differentiation factor, eosinophil differentiation factor, vascular endothelial growth factor (VEGF), fibroblast growth factor (FGF)-3, FGF-4, FGF-5, FGF-6, FGF-7, FGF-8, FGF-9, basic fibroblast growth factor, platelet-induced growth factor, transforming growth factor beta 1, acidic fibroblast growth factor, osteonectin, angiopoietin 1, angiopoietin 2, insulin-like growth factor, platelet-derived growth factor AA, platelet-derived growth factor BB, platelet-derived growth factor AB, endothelial PAS protein 1, thrombospondin, proliferin, Ephrin-A1, E-selectin, leptin, heparin, thyroxine, and sphingosine 1-phosphate. In other embodiments, the second biologically active compound may comprise other medications and compounds, including without limitation various cytokines (including growth factors) and antibodies (discussed below). Representative cytokines which may function as one or more second biologically active compound second biologically active compounds include, by way of example and not of limitation, beta interferon, gamma interferon, tumor necrosis factor, and any of the growth factors discussed above.

In accordance with the present invention, local delivery of a growth factor, via elution from layer 52 of the medical device 10, provides a therapeutic strategy and method of treatment of tissue damage by promoting engraftment and differentiation of hematopoietic stem cells and/or endothelial progenitor cells at the site of (myocardial or arterial) injury. There is emerging direct and indirect evidence that the recovery of myocardial function, after an acute MI or in chronically severely ischemic myocardium, is linked to the regeneration and differentiation potential of hematopoietic stem cells (HSC) and/or endothelial progenitor cells. Stem or progenitor cell migration, engraftment and differentiation should occur at the site of myocardial damage, which invariably is downstream from the site of arterial occlusion or critical narrowing (which is the target site for stent placement). Biologic therapies derived from transplantation of such hematopoietic stem cells and/or endothelial progenitor cells, through tissue regeneration and repair as well as through the targeted delivery of genetic material, can be effective in the treatment of a wide range of medical conditions. After transplantation, these cells can differentiate into different tissue types depending upon the microenvironment and availability of growth factors, including cardiac myocytes, endothelial cells, and other vascular and neural tissues. Although cardiac myocytes have been considered as terminally differentiated cells, it has been recently reported that bone marrow stem cells (BMSCs) can differentiate into cardiac myocytes and endothelial cells which may result in regeneration of cardiac myocytes and blood vessels. In accordance with the invention, cytokine-mediated regeneration therapy is also a novel therapeutic strategy for myocardial injury. While described with respect to myocardial injury, it will be understood that this methodology extends to treatment of other forms of tissue damage, which are also within the scope of the present invention. In addition, while described with respect to hematopoietic stem cells and endothelial progenitor cells, it should be understood that the use of growth factors within the scope of the present invention extends to aiding the differentiation, engraftment, and proliferation of any type of progenitor or other stem cell.

Several hematopoietic, chematopoietic, or other growth factors, including interleukin-3 (IL-3), IL-6, granulocyte-macrophage colony-stimulating factors (GM-CSF), granulocyte colony-stimulating factor (G-CSF), and stem cell factor (SCF) have been reported to regulate different stages of stem cell development. GCSF plays a role in the regulation of proliferation, differentiation, and survival of hematopoietic stem cells and/or other progenitor cells. GCSF also causes a marked increase in the release of hematopoietic stem cells (HSCs) and/or other progenitor cells into the peripheral blood circulation, a process referred to as stem cell mobilization.

Conventional methods of mobilization of the stem or other progenitor cells for transplant applications involve systemic administration of growth factors such as G-CSF and GM-CSF. However, these conventional methods are also associated with systemic side effects resulting from these growth factors. The present invention incorporates a novel method of local delivery of these growth factors and other cytokines at the tissue injury site, providing for increased in vivo expansion, differentiation and survival of the transplanted, mobilized or recruited stem cells. Studies done with the intra coronary infusion or intra myocardial injection of these stem cells have shown that 90% of these cells are dead within 48 hours of infusion. Local sustained delivery of the growth factors and cytokines, in accordance with the invention, will result in better survival and in vivo expansion in the end organ (myocardium) microenvironment. This will improve the engraftment and differentiation of the stem cells to different tissue types.

When the medical device 10 has been inserted at its target site, the second biologically active compound or combination of compounds is delivered via the flow of blood to downstream targets in the myocardial or other microenvironments. In accordance with the present invention, this downstream delivery to second target sites of one or more growth factors promotes the viability, engraftment and eventual differentiation of progenitor stem cells or immature cells that are either injected directly into the coronary bed, after autologous harvesting from a peripheral blood source (apheresis) or bone marrow source such as the iliac crest, or which are autonomously recruited from the circulating pool of premature cells. One or more of these drugs, as the one or more second biologically active compounds, also facilitates the endothelialization of the stent at the first, local site of stent placement.

The hematopoietic stem cells are capable of differentiating into cardiac myocytes, thereby regenerating the damaged myocardium after an acute myocardial infarction or in critically and chronically ischemic myocardium. The endothelial cells, which line blood vessels, contain receptors that bind to growth factors. Binding of the growth factors to these receptors triggers a complex series of events, including the replication and migration of endothelial cells to ischemic sites, as well as their formation into new blood vessels. However, in ischemic conditions, the growth factor genes often may not produce sufficient amounts of the corresponding proteins to generate an adequate number of new blood vessels. In accordance with the present invention, a therapeutic approach to this problem is to enhance the body's own response by temporarily providing higher concentrations of growth factors at the second, target site of injury or other disease.

For cardiac disease, this will require a cardiovascular delivery system. Conventional systemic administration of the growth factors results in systemic side effects, such as a steep increase in the white blood cell count which increases the viscosity of the blood stream, thereby interfering with blood flow. In contrast, the present invention provides a method of localized, in vivo delivery of growth factors, thereby preventing such systemic side effects.

Once the stent has been inserted, this second biologically active compound may be released into the arterial lumen at the site of the stent structure 20, as well as delivered via the bloodstream to targets downstream from the site of stent implantation, such as the myocardial environment. The second biologically active compound is released by physical dissociation, aqueous solubility, protein binding, or by biodegradation or bioresorption of any of the layers of the biologically active structure 60 into the bloodstream. Polymer layer 52 containing the second biologically active compound, such as in the form of a polymeric matrix, may degrade or dissolve either by enzymatic hydrolysis, by exposure to water within plasma or other fluids, or by surface or bulk erosion.

The delivery of the second biologically active compound to the site of stent implantation and further downstream to a second site serves two purposes. First, the delivery facilitates the endothelialization of the stent at the site of stent implantation. Second, the delivery of the second biologically active compound to the downstream myocardial microenvironment, as a second site, promotes in vivo expansion, viability, engraftment, differentiation, and maturation of progenitor stem cells or immature cells. As indicated above, these cells are either injected directly into the coronary bed, after autologous harvesting from a peripheral blood source by a procedure of apheresis or a bone marrow source such as the iliac crest, or are autonomously recruited from the circulating pool of premature cells.

The growth factor or combination of growth factors may be released post-MI immediately following implantation of the medical device. Release may be sustained for an extended period of time. To obtain different release times and rates of release, the layers 50, 52 (and any sublayers or polymeric matrix) may be formed of different polymers or the same polymer with different degrees of crosslinking. Furthermore, the ratio of the polymer to the second biologically active compound can be varied. When this ratio is higher, the second biologically active compound is released more slowly into the lumen.

In another embodiment of the present invention, one or more of the second biologically active compounds may be one or more antibodies, such as an antibody having a binding affinity for CD34 receptors of hematopoietic stem cells and/or endothelial progenitor cells. More specifically, a layer of such chemotactic, antibody material may be incorporated in layer 52 (generally on its surface) or otherwise layered onto the polymeric matrix comprised of a polymer and one or more other second biologically active compounds (and forming layer 52). Antibodies which have a (selective) binding affinity to CD34 receptors may be covalently or noncovalently coated on the polymeric matrix after application of the polymeric matrix to the stent structure 20. This chemotactic layer increases the probability of attachment of CD34 cells to the inner surface 22 of the stent structure 20 so as to promote the formation of an endothelial layer on the inner surface 22 of the stent structure 20. CD34 receptors are glycoproteins found on immature hematopoietic cells. Other antibodies and antibody fragments within the scope of the present invention, in addition to those which have a binding affinity for CD34 receptors, are discussed below.

As used herein, the term “antibody” refers to any type of monoclonal, polyclonal, humanized, or chimeric antibody or a combination or fragment thereof, wherein the monoclonal, polyclonal, humanized or chimeric antibody binds to one antigen or a functional equivalent of that antigen, which, in this case, is a binding affinity to one or more of the following antigens: CD34, CD133, CD34, CDw90, CD117, HLA-DR, VEGFR-1, VEGFR-2, Muc-18 (CD146), CD130, stem cell antigen (Sca-1), stem cell factor 1 (SCF/c-Kit ligand), Tie-2 and HAD-DR. The term antibody fragment encompasses any fragment of an antibody such as Fab, F(ab′).sub.2, and can be of any size, i.e., large or small molecules, which have the same results or effects as the antibody. (An antibody encompasses a plurality of individual antibody molecules equal to 6.022·10²³ molecules per mole of antibody). The antibodies of the present invention recognize and bind progenitor hematopoietic stem cell, endothelial progenitor cell and/or other progenitor or stem cell surface antigens in the circulating blood so that the cells are immobilized on the inner, luminal (28) surface of the medical device 10.

In the selected embodiments, the biologically active structure 60 includes an impermeable layer 40 that is capable of limiting exposure of the first biologically active compound (located on or exposed to the outer surface of the device 10) primarily or exclusively to the surrounding vascular tissue, when the device has been implanted at its target location (such as an artery). This impermeable layer 40 also limits the unintended delivery of the first biologically active compound from the outer surface of the device 10 to the downstream site of myocardial tissue injury or chronic critical ischemia. In addition, this impermeable layer 40 also limits the release of the second biologically active compound (located on or exposed to the inner surface of device 10) primarily or exclusively to the bloodstream (and downstream targets) and significantly prevents release of the second biologically active compound to the surrounding vascular tissue. These biologically active compounds are thereby independently and selectively delivered to two (or more) separate target sites, at different locations, in order to both prevent restenosis and promote tissue regeneration. First, anti-proliferative, cytostatic, or cytotoxic drugs are delivered to the vascular tissue surrounding the implanted medical device 10, and are concomitantly prevented from being delivered to other sites, such as the site of myocardial injury, where these compounds could impair or inhibit tissue regeneration and healing. Second, a growth factor or combination of growth factors may be delivered, selectively and independently, and at any time, to the arterial lumen, for activity at a second site.

Depending on the selected embodiment, when one or more second biologically active compounds (such growth factors) may be inappropriate or undesired, such second biologically active compounds may not be and are not required to be included in the device 10. Similarly, under various circumstances, one or more first biologically active compounds may be inappropriate or undesired, and as a consequence, such first biologically active compounds may not be and are not required to be included in the device 10. In these cases, only one of the halves of biologically active structure 60 may be included in the device 10, either biologically active structure 62 or 64. For example, a growth factor stent is within the scope of the present invention, in which a growth factor is included as the second biologically active compound in layer 52 of biologically active structure 64, with any inclusion of biologically active structure 62 being optional.

The medical device 10 may be provided in a method capable of preventing restenosis of an implantable medical device and capable of aiding in endothelialization of the implantable medical device. In order to prevent restenosis, aid in endothelialization, and help in myocardial regeneration, two biologically active compounds may be utilized, simultaneously, sequentially, or both, which have mutually antagonistic activities. To the extent of such mutually antagonistic activities, or for any other reason for which it is advisable to limit exposure to one of the biologically active compounds, the insulation of each biologically active compound is significant. Such insulation of one biologically active compound from another biologically active compound provides the capability for selective and independent effectiveness of each, without interference and/or inhibition from the other, providing for each to have their corresponding potential beneficial effects. For example, if the anti-proliferative, cytostatic, or cytotoxic biologically active compound were not insulated from release via impermeable layer 40 and instead were released into the lumen of the artery, the hematopoietic stem cells and/or endothelial progenitor cells would be damaged, thereby delaying or even preventing endothelialization and myocardial regeneration.

Similarly, it is important that the growth factor released in the arterial lumen, capable of aiding in endothelialization and myocardial regeneration, be insulated from the vascular tissue surrounding the medical device 10. If the growth factor were not insulated from release via impermeable layer 40 and instead were released into the vascular tissue surrounding the outer surface of the medical device 10, neointimal hyperplasia and restenosis may be induced, an action directly adverse to and opposing the action of the anti-proliferative, cytostatic, or cytotoxic drug designed for delivery into the vascular tissue. A method is therefore provided capable of performing two distinct functions by utilizing, and insulating each from the other, two biologically active compounds with mutually antagonistic biological activities.

A method of treatment of myocardial tissue is also provided in which a first biologically active compound that is anti-proliferative, cytostatic, or cytotoxic, and capable of preventing restenosis, is delivered to a first target site, and a second biologically active compound, comprising a growth factor capable of promoting repair, generation, or regeneration of damaged myocardial tissue, is delivered to a second target site. The first target site includes the site of implantation or placement of the medical device, while the second target site is an area of tissue injury, such as the area of myocardial tissue damaged by an MI. The second biologically active compound reaches the second target site by delivery into the arterial lumen and bloodstream, so that the bloodstream transports the second biologically active compound to the second target site.

Again, the insulation of each biologically active compound from the other's opposing action is important in order to assure its independent effect without interference and inhibition from the other biologically active compound. For example, if the first biologically active compound, capable of preventing restenosis, were released into the lumen of the artery and delivered further downstream, it may be potentially harmful to the stem cells and premature progenitor cells as they are attempting to engraft, differentiate, and mature in the myocardial microenvironment downstream from the site of the medical device. The endothelial progenitor stem cells also would be damaged, delaying endothelialization and potentially causing stent thrombosis.

Similarly, it is significant that the second biologically active compound, such as a growth factor, which is released in the arterial lumen, is also insulated from release into the vascular tissue surrounding the outer surface of the medical device. The release of such growth factors may induce neointimal hyperplasia and restenosis, an action directly adverse to the action of the anti-proliferative, cytotoxic, and cytostatic biologically active compound provided for delivery into the vascular tissue. A method of treatment is therefore claimed whereby two distinct functions are performed by the utilization and insulation of two biologically active compounds with mutually antagonistic biological activities.

In summary, the present invention provides a medical device 10 comprising a stent structure 20 and a biologically active structure 60 attached to the stent structure 20. The stent structure 20 has an outer surface 22 and an inner surface 24 that defines a lumen 28. The biologically active structure 60 has a plurality of layers, a first layer 50 of the plurality of layers having a first biologically active compound with a first biological activity, a second layer 52 of the plurality of layers having a second biologically active compound having a second biological activity, and a third layer 40 of the plurality of layers located between the first and second layers, wherein the third layer 40 is impermeable to the first biologically active compound and to the second biologically active compound. The second biological activity may be and often is antagonistic to the first biological activity.

The first layer 50 and the second layer 52 of the biologically active structure may be comprised of a plurality of sublayers. A first sublayer of the plurality of sublayers is a first polymer having either the first biologically active compound or the second biologically active compound, and a second sublayer of the plurality of sublayers is a second polymer having a predetermined release rate, respectively, for the first biologically active compound or for the second biologically active compound.

Also in summary, the present invention provides a medical device 10 for drug delivery, comprising a stent structure 20 and a biologically active structure 62, 64. The stent structure 20 has an outer surface 20 and an inner surface 24 that defines a lumen 28. The biologically active structure 62, 64 has a plurality of layers, a first layer (50 or 52) of the plurality of layers having a biologically active compound with a biological activity, and a second layer 40 of the plurality of layers which is attached to the stent structure 20, wherein the second layer is impermeable to the biologically active compound. This second layer may be attached to either or both the outer surface and inner surface of the stent structure, respectively forming biologically active structure 62 or 64.

While the invention has been particularly shown and described with reference to the exemplary embodiments thereof, it is well known by those skilled in the art that various changes and modifications can be made in the invention without departing from the spirit and scope of the invention. The present invention is not restricted to the particular constructions described and illustrated, but should be constructed to include and cohere with all modifications that may fall within the scope of the appended claims.

1. A medical device comprising:
a stent structure having an outer surface and an inner surface that defines a lumen; and
a biologically active structure attached to the stent structure, the biologically active structure having a plurality of layers, a first layer of the plurality of layers having a first biologically active compound with a first biological activity, a second layer of the plurality of layers having a second biologically active compound having a second biological activity, and a third layer of the plurality of layers located between the first and second layers, wherein the third layer is substantially impermeable to the first biologically active compound and to the second biologically active compound.

2. The medical device of claim 1 wherein the second biological activity is substantially antagonistic to the first biological activity.

3. The medical device of claim 1 wherein the stent structure is comprised of one or more of the following materials: stainless steel, a nickel-titanium alloy, a cobalt-chromium alloy, a magnesium alloy, carbon, carbon fiber, or a polymer.

4. The medical device of claim 1 wherein the third layer is a membrane.

5. The medical device of claim 4 wherein the membrane is elastomeric, biocompatible, non-allergenic, and non-thrombotic.

6. The medical device of claim 4 wherein the membrane is comprised of polytetrafluoroethylene.

7. The medical device of claim 1 wherein the plurality of layers are elastomeric.

8. The medical device of claim 7 wherein the plurality of layers are biocompatible, non-allergenic, and non-thrombotic.

9. The medical device of claim 8 wherein each of the first, second and third layers of the plurality of layers is a polymer.

10. The medical device of claim 9 wherein elution kinetics of either the first biologically active compound or the second biologically active compound are predetermined based on the ratio of polymer to, respectively, either the first biologically active compound or the second biologically active compound.

11. The medical device of claim 9 wherein the polymer is selected from a plurality of polymers, the plurality of polymers comprising one or more of the following polymers, their respective derivatives and copolymers: poly(ethers), poly(ethylene oxide), poly(ethylene glycol), poly(tetramethylene oxide); vinyl polymers, poly(acrylates), poly(methacrylates) such as methyl, ethyl, other alkyl, hydroxyethyl methacrylate, acrylic acids, methacrylic acids, poly(vinyl alcohol), poly(vinyl pyrolidone), poly(vinyl acetate); poly(urethanes); cellulose and its derivatives s alkyl, hydroxyalkyl, ethers, esters, nitrocellulose, cellulose acetates; poly(siloxanes); plasticized nylon, plasticized soft nylon, natural rubber, silicone, medical grade silicone rubbers, ethylene-propylene rubber, silicone-carbonate copolymers, poly(olefins, poly(vinyl-olefins), poly(styrene), poly(halo-olefins), poly(isobutylene), polylactide, polylactide-co-glycolide, polydioxanone, thermoplastic elastomers, thermpoplastics, expanded PTFE, poly(vinyl-chloride), poly(isoprene), poly(isobutylene), poly(butadiene), polymalic acid, polyamino acids, polyacrylic acids, polyethylene glycol, polyvinylpyrrolidone, polyvinyl alcohols, hydrophilic polyurethanes, albumin, collagen, gelatin, starch, cellulose, dextran, polymalic acid, polyamino acids and their co-polymers or lightly cross-linked forms, polysaccharides and their derivatives, sodium alginate, karaya gum, gelatin, guar gum, agar, align, carrageenans, pectin, locust bean gums, xanthan, starch-based gums, hydroxyalkyl and ethyl ethers of cellulose, sodium carboxymethylcellulose, poly(amides) such as poly(amino acids) and poly(peptides); poly(esters) such as poly(lactic acid), poly(glycolic acid), poly(lactic-co-glycolic acid), and poly(caprolactone);
poly(anhydrides); poly(orthoesters); poly(carbonates).

12. The medical device of claim 1 wherein the third layer is comprised of one or more of the following: ethylene vinyl acetate, latexes, urethanes, polysiloxanes, styrene-ethylene block copolymers, butylene-styrene block copolymers, silicone rubber, Silastic, and aliphatic polyesters, and mixtures and copolymers thereof.

13. The medical device of claim 1 wherein the biologically active structure is attached to the outer surface of the stent structure.

14. The medical device of claim 1 wherein the biologically active structure is attached to the inner surface of the stent structure.

15. The medical device of claim 1 wherein the biologically active structure is interleaved with the stent structure.

16. The medical device of claim 1 wherein the biologically active structure is attached to both the inner surface of the stent structure and the outer surface of the stent structure.

17. The medical device of claim 1 wherein the first layer and the second layer of the biologically active structure are each comprised of a plurality of sublayers.

18. The medical device of claim 17 wherein a first sublayer of the plurality of sublayers is a first polymer having either the first biologically active compound or the second biologically active compound, and wherein a second sublayer of the plurality of sublayers is a second polymer having a predetermined release rate, respectively, for the first biologically active compound or for the second biologically active compound.

19. The medical device of claim 17 wherein the first sublayer comprises a copolymer of ethylene-co-vinylacetate and polybutylmethacrylate and the second sublayer is polybutylmethacrylate.

20. The medical device of claim 1 wherein the first biological activity is a first pharmacological effect and the second biological activity is a second pharmacological effect, wherein the first pharmacological effect is opposing and adverse to the second pharmacological effect.

21. The medical device of claim 1 wherein the first biological activity is a first pharmacological effect and the second biological activity is a second pharmacological effect, wherein the first pharmacological effect interferes with the second pharmacological effect.

22. The medical device of claim 1 wherein the first biological activity is anti-proliferative.

23. The medical device of claim 1 wherein the first biological activity is anti-inflammatory.

24. The medical device of claim 1 wherein the first biological activity is either cytostatic or cytotoxic.

25. The medical device of claim 1 wherein the first biologically active compound is one or more of the following and analogues thereof: rapamycin, heparin, anti-thrombin compounds, prostaglandin inhibitors, platelet inhibitors, taxol, taxol derivatives, tacrolimus, tachrolimus-containing compounds, cytochalasin, paclitaxel, dexamethasone, a steroid compound, methotrexate, prednisolone, corticosterone, budesonide, estrogen, sulfasalazine, mesalamine, 5-fluorouracil, cisplatin, vinblastine, vincristine, epothilones, endostatin, angiostatin, tyrosine kinase inhibitors, lidocaine, bupivacaine, ropivacaine.

26. The medical device of claim 1 wherein the second biologically active compound is a growth factor.

27. The medical device of claim 26 wherein the growth factor is one or more of the following: granulocyte colony-stimulating factor (G-CSF), granulocyte-macrophage colony-stimulating factor (GM-CSF), CSF-1, G-CSF Ser. sup. 17, M-CSF, c-mpl ligand (MGDF or TPO), erythropoietin (EPO), stem cell factor (SCF), interleukins 1-16 (IL-1, IL-2, IL-3, IL-4, IL-5, IL-6, IL-7, IL-8, IL-9, IL-10, IL-11, IL-12, IL-13, IL-14, IL-15, IL-16), flt3 ligand, human growth hormone, B-cell growth factor, B-cell differentiation factor, eosinophil differentiation factor, vascular endothelial growth factor (VEGF), fibroblast growth factor (FGF)-3, FGF-4, FGF-5, FGF-6, FGF-7, FGF-8, FGF-9, basic fibroblast growth factor, platelet-induced growth factor, transforming growth factor beta 1, acidic fibroblast growth factor, osteonectin, angiopoietin 1, angiopoietin 2, insulin-like growth factor, platelet-derived growth factor AA, platelet-derived growth factor BB, platelet-derived growth factor AB, endothelial PAS protein 1, thrombospondin, proliferin, Ephrin-A1, E-selectin, leptin, heparin, thyroxine, sphingosine 1-phosphate.

28. The medical device of claim 1 wherein the second biologically active compound is a cytokine.

29. The medical device of claim 28 wherein the cytokine is one or more of the following: a growth factor, beta interferon, gamma interferon, tumor necrosis factor.

30. The medical device of claim 1 wherein the second layer further comprises an antibody or antibody fragment.

31. The medical device of claim 30 wherein the antibody or antibody fragment has a binding affinity to one or more of the following: CD34 receptors, CD133 receptors, CDw90 receptors, CD117 receptors, HLA-DR, VEGFR-1, VEGFR-2, Muc-18 (CD146), CD130, stem cell antigen (Sca-1), stem cell factor 1 (SCF/c-Kit ligand), Tie-2, HAD-DR.

32. The medical device of claim 1 wherein the biologically active structure spans an entire length and entire circumference of the stent structure.

33. The medical device of claim 1 wherein the biologically active structure spans a partial length of the stent structure.

34. The medical device of claim 1 wherein the biologically active structure spans a partial circumference of the stent structure.

35. The medical device of claim 1 wherein a degree of impermeability of the third layer is selected based upon pharmacological sufficiency.

36. The medical device of claim 35 wherein the pharmacological sufficiency is determined by one or more of the following factors: selected purposes of the first second biologically active compound and the second biologically active compound; pharmacological effects of the first second biologically active compound and the second biologically active compound; medical treatment objectives; medical treatment durations; a rate of degradation or metabolism of the first second biologically active compound and the second biologically active compound and their active metabolites; a degree of acceptable elution without significantly adverse effects; a rate of bioresorption of the first layer or the second layer.

37. The medical device of claim 1 wherein, when the medical device is placed in an artery abutting vascular tissue, the first layer is capable of selectively releasing the first biologically active compound into the vascular tissue and the second layer is capable of selectively and independently releasing the second biologically active into an arterial lumen.

38. The medical device of claim 1 wherein the third layer is selectively impermeable.

39. A method of forming an implantable medical device, the implantable medical device capable of preventing restenosis and preventing thrombosis by aiding endothelialization of the implantable medical device, the method comprising:
providing a stent including an impermeable layer capable of substantially insulating a first biologically active compound from a second biologically active compound;
incorporating the first biologically active compound within a polymeric matrix on a first surface of the impermeable layer, wherein the first biologically active compound is capable of substantially preventing restenosis; and
incorporating a second biologically active compound within a polymeric matrix on a second surface of the impermeable layer, wherein the second biologically active compound is capable of substantially preventing thrombosis by substantially aiding endothelialization of the stent.

40. A method of treatment of myocardial tissue, capable of promoting the regeneration of myocardial cells, comprising the steps of:
inserting a stent into a first target site, the stent having a first biologically active compound having a first biological activity and having a second biologically active compound having a second, substantially antagonistic biologically activity, wherein the first biologically active compound is substantially insulated from the second biologically active compound;
delivering locally and selectively the first biologically active compound to the first target site; and
delivering the second biologically active compound via the bloodstream to a second target site.

41. The method of claim 40 wherein the second target site is myocardial tissue.

42. The method of claim 40 wherein the first biologically active compound is anti-proliferative and the second biologically active compound is a growth factor.

43. The method of claim 42 wherein the growth factor is substantially capable of promoting regeneration of myocardial tissue by aiding engraftment and differentiation of hematopoietic stem cells.

44. The method of claim 42 wherein the growth factor is substantially capable of promoting regeneration of myocardial tissue by aiding engraftment and differentiation of endothelial progenitor cells.

45. The method of claim 42 wherein the growth factor is one or more of the following: granulocyte colony-stimulating factor (G-CSF), granulocyte-macrophage colony-stimulating factor (GM-CSF), CSF-1, G-CSF Ser. sup. 17, M-CSF, c-mpl ligand (MGDF or TPO), erythropoietin (EPO), stem cell factor (SCF), interleukins 1-16 (IL-1, IL-2, IL-3, IL-4, IL-5, IL-6, IL-7, IL-8, IL-9, IL-10, IL-11, IL-12, IL-13, IL-14, IL-15, IL-16), flt3 ligand, human growth hormone, B-cell growth factor, B-cell differentiation factor, eosinophil differentiation factor, vascular endothelial growth factor (VEGF), fibroblast growth factor (FGF)-3, FGF-4, FGF-5, FGF-6, FGF-7, FGF-8, FGF-9, basic fibroblast growth factor, platelet-induced growth factor, transforming growth factor beta 1, acidic fibroblast growth factor, osteonectin, angiopoietin 1, angiopoietin 2, insulin-like growth factor, platelet-derived growth factor AA, platelet-derived growth factor BB, platelet-derived growth factor AB, endothelial PAS protein 1, thrombospondin, proliferin, Ephrin-A1, E-selectin, leptin, heparin, thyroxine, sphingosine 1-phosphate.

46. The method of claim 42 wherein the second biologically active compound further comprises an antibody or antibody fragment.

47. The method of claim 46 wherein the antibody or antibody fragment has a binding affinity to one or more of the following: CD34 receptors, CD133 receptors, CDw90 receptors, CD117 receptors, HLA-DR, VEGFR-1, VEGFR-2, Muc-18 (CD146), CD130, stem cell antigen (Sca-1), stem cell factor 1 (SCF/c-Kit ligand), Tie-2, HAD-DR.

48. The method of claim 40 wherein the second biologically active compound is a cytokine.

49. The method of claim 48 wherein the cytokine is one or more of the following: a growth factor, beta interferon, gamma interferon, tumor necrosis factor.

50. A medical device comprising
a stent structure having an outer surface and an inner surface that defines a lumen; and
a biologically active structure attached to the stent structure, the biologically active structure having a plurality of layers, a first layer of the plurality of layers having a first biologically active compound with a first biological activity, a second layer of the plurality of layers having a second biologically active compound having a second biological activity, and a third layer of the plurality of layers located between the first and second layers, wherein the second biological activity is substantially antagonistic to the first biological activity.

51. The medical device of claim 50 wherein the third layer is substantially impermeable to the first biologically active compound and to the second biologically active compound.

52. The medical device of claim 51 wherein the third layer is a biocompatible, non-allergenic, and non-thrombotic membrane comprised of polytetrafluoroethylene.

53. The medical device of claim 50 wherein the third layer is selectively impermeable to the first biologically active compound and to the second biologically active compound.

54. The medical device of claim 50 wherein the plurality of layers are one or more elastomeric, biocompatible, non-allergenic, and non-thrombotic polymers.

55. The medical device of claim 50 wherein elution kinetics of either the first biologically active compound or the second biologically active compound are predetermined based on the ratio of polymer to, respectively, either the first biologically active compound or the second biologically active compound.

56. The medical device of claim 50 wherein the first layer and the second layer of the biologically active structure are each comprised of a plurality of sublayers.

57. The medical device of claim 56 wherein a first sublayer of the plurality of sublayers is a first polymer having either the first biologically active compound or the second biologically active compound, and wherein a second sublayer of the plurality of sublayers is a second polymer having a predetermined release rate, respectively, for the first biologically active compound or for the second biologically active compound.

58. The medical device of claim 56 wherein the first sublayer comprises a copolymer of ethylene-co-vinylacetate and polybutylmethacrylate and the second sublayer is polybutylmethacrylate.

59. The medical device of claim 50 wherein the first biological activity is anti-proliferative and anti-inflammatory.

60. The medical device of claim 50 wherein the first biological activity is either cytostatic or cytotoxic.

61. The medical device of claim 50 wherein the first biologically active compound is one or more of the following: rapamycin, heparin, anti-thrombin compounds, prostaglandin inhibitors, platelet inhibitors, taxol, taxol derivatives, tacrolimus, tachrolimus-containing compounds, cytochalasin, paclitaxel, dexamethasone, a steroid compound, methotrexate, prednisolone, corticosterone, budesonide, estrogen, sulfasalazine, mesalamine, 5-fluorouracil, cisplatin, vinblastine, vincristine, epothilones, endostatin, angiostatin, tyrosine kinase inhibitors, lidocaine, bupivacaine, ropivacaine.

62. The medical device of claim 50 wherein the second biologically active compound is a growth factor.

63. The medical device of claim 62 wherein the growth factor is one or more of the following: granulocyte colony-stimulating factor (G-CSF), granulocyte-macrophage colony-stimulating factor (GM-CSF), CSF-1, G-CSF Ser. sup. 17, M-CSF, c-mpl ligand (MGDF or TPO), erythropoietin (EPO), stem cell factor (SCF), interleukins 1-16 (IL-1, IL-2, IL-3, IL-4, IL-5, IL-6, IL-7, IL-8, IL-9, IL-10, IL-11, IL-12, IL-13, IL-14, IL-15, IL-16), flt3 ligand, human growth hormone, B-cell growth factor, B-cell differentiation factor, eosinophil differentiation factor, vascular endothelial growth factor (VEGF), fibroblast growth factor (FGF)-3, FGF-4, FGF-5, FGF-6, FGF-7, FGF-8, FGF-9, basic fibroblast growth factor, platelet-induced growth factor, transforming growth factor beta 1, acidic fibroblast growth factor, osteonectin, angiopoietin 1, angiopoietin 2, insulin-like growth factor, platelet-derived growth factor AA, platelet-derived growth factor BB, platelet-derived growth factor AB, endothelial PAS protein 1, thrombospondin, proliferin, Ephrin-A1, E-selectin, leptin, heparin, thyroxine, sphingosine 1-phosphate.

64. The medical device of claim 50 wherein the second layer further comprises an antibody or antibody fragment. having a binding affinity to CD34 receptors of hematopoietic stem cells.

65. The medical device of claim 64 wherein the antibody or antibody fragment has a binding affinity to one or more of the following: CD34 receptors, CD133 receptors, CDw90 receptors, CD117 receptors, HLA-DR, VEGFR-1, VEGFR-2, Muc-18 (CD146), CD130, stem cell antigen (Sca-1), stem cell factor 1 (SCF/c-Kit ligand), Tie-2, HAD-DR.

66. The medical device of claim 50 wherein the second biologically active compound is a cytokine.

67. The medical device of claim 66 wherein the cytokine is one or more of the following: a growth factor, beta interferon, gamma interferon, tumor necrosis factor.

68. A medical device for drug delivery, comprising
a stent structure having an outer surface and an inner surface that defines a lumen; and
a biologically active structure attached to the stent structure, the biologically active structure having a plurality of layers, a first layer of the plurality of layers having a biologically active compound with a biological activity, and a second layer of the plurality of layers being substantially impermeable to the biologically active compound.

69. The medical device of claim 68 wherein the second layer is attached to the outer surface of the stent structure, or wherein the first layer is attached to the inner surface of the stent structure, or wherein the first layer is attached to the outer surface of the stent structure and the second layer is attached to the inner surface of the stent structure.

70. The medical device of claim 69 wherein the biological activity is anti-proliferative and anti-inflammatory.

71. The medical device of claim 69 wherein the first biological activity is either cytostatic or cytotoxic.

72. The medical device of claim 69 wherein the first biologically active compound is one or more of the following: rapamycin, heparin, anti-thrombin compounds, prostaglandin inhibitors, platelet inhibitors, taxol, taxol derivatives, tacrolimus, tachrolimus-containing compounds, cytochalasin, paclitaxel, dexamethasone, a steroid compound, methotrexate, prednisolone, corticosterone, budesonide, estrogen, sulfasalazine, mesalamine, 5-fluorouracil, cisplatin, vinblastine, vincristine, epothilones, endostatin, angiostatin, tyrosine kinase inhibitors, lidocaine, bupivacaine, ropivacaine.

73. The medical device of claim 68 wherein the second layer is attached to the inner surface of the stent structure, or wherein the first layer is attached to the outer surface of the stent structure, or wherein the first layer is attached to the inner surface of the stent structure and the second layer is attached to the outer surface of the stent structure.

74. The medical device of claim 73 wherein the biologically active compound is a growth factor.

75. The medical device of claim 73 wherein the growth factor is one or more of the following: granulocyte colony-stimulating factor (G-CSF), granulocyte-macrophage colony-stimulating factor (GM-CSF), CSF-1, G-CSF Ser. sup. 17, M-CSF, c-mpl ligand (MGDF or TPO), erythropoietin (EPO), stem cell factor (SCF), interleukins 1-16 (IL-1, IL-2, IL-3, IL-4, IL-5, IL-6, IL-7, IL-8, IL-9, IL-10, IL-11, IL-12, IL-13, IL-14, IL-15, IL-16), flt3 ligand, human growth hormone, B-cell growth factor, B-cell differentiation factor, eosinophil differentiation factor, vascular endothelial growth factor (VEGF), fibroblast growth factor (FGF)-3, FGF-4, FGF-5, FGF-6, FGF-7, FGF-8, FGF-9, basic fibroblast growth factor, platelet-induced growth factor, transforming growth factor beta 1, acidic fibroblast growth factor, osteonectin, angiopoietin 1, angiopoietin 2, insulin-like growth factor, platelet-derived growth factor AA, platelet-derived growth factor BB, platelet-derived growth factor AB, endothelial PAS protein 1, thrombospondin, proliferin, Ephrin-A1, E-selectin, leptin, heparin, thyroxine, and sphingosine 1-phosphate.

76. The medical device of claim 73 wherein the biologically active compound is an antibody or antibody fragment.

77. The medical device of claim 76 wherein the antibody or antibody fragment has a binding affinity to one or more of the following: CD34 receptors, CD133 receptors, CDw90 receptors, CD117 receptors, HLA-DR, VEGFR-1, VEGFR-2, Muc-18 (CD146), CD130, stem cell antigen (Sca-1), stem cell factor 1 (SCF/c-Kit ligand), Tie-2, HAD-DR.

78. The medical device of claim 73 wherein the biologically active compound is a cytokine.

79. The medical device of claim 78 wherein the cytokine is one or more of the following: a growth factor, beta interferon, gamma interferon, tumor necrosis factor.

80. The medical device of claim 68 wherein the plurality of layers are elastomeric, biocompatible, non-allergenic, and non-thrombotic polymer.

81. The medical device of claim 68 wherein the first layer of the biologically active structure is comprised of a plurality of sublayers.

82. The medical device of claim 81 wherein a first sublayer of the plurality of sublayers is a first polymer having the biologically active compound, and wherein a second sublayer of the plurality of sublayers is a second polymer having a predetermined release rate for the biologically active compound.

83. The medical device of claim 82 wherein the first sublayer comprises a copolymer of ethylene-co-vinylacetate and polybutylmethacrylate and the second sublayer is polybutylmethacrylate.

84. A medical device for drug delivery, comprising
a stent structure having an outer surface and an inner surface that defines a lumen; and
a biologically active structure attached to the stent structure, the biologically active structure having a plurality of layers, a first layer of the plurality of layers having a growth factor, and a second layer of the plurality of layers being substantially impermeable to the growth factor.

85. The medical device of claim 84 wherein the growth factor is one or more of the following: granulocyte colony-stimulating factor (G-CSF), granulocyte-macrophage colony-stimulating factor (GM-CSF), CSF-1, G-CSF Ser. sup. 17, M-CSF, c-mpl ligand (MGDF or TPO), erythropoietin (EPO), stem cell factor (SCF), interleukins 1-16 (IL-1, IL-2, IL-3, IL-4, IL-5, IL-6, IL-7, IL-8, IL-9, IL-10, IL-11, IL-12, IL-13, IL-14, IL-15, IL-16), flt3 ligand, human growth hormone, B-cell growth factor, B-cell differentiation factor, eosinophil differentiation factor, vascular endothelial growth factor (VEGF), fibroblast growth factor (FGF)-3, FGF-4, FGF-5, FGF-6, FGF-7, FGF-8, FGF-9, basic fibroblast growth factor, platelet-induced growth factor, transforming growth factor beta 1, acidic fibroblast growth factor, osteonectin, angiopoietin 1, angiopoietin 2, insulin-like growth factor, platelet-derived growth factor AA, platelet-derived growth factor BB, platelet-derived growth factor AB, endothelial PAS protein 1, thrombospondin, proliferin, Ephrin-A1, E-selectin, leptin, heparin, thyroxine, sphingosine 1-phosphate.

86. The medical device of claim 84 wherein the first layer further comprises an antibody or antibody fragment having a binding affinity to one or more of the following: CD34 receptors, CD133 receptors, CDw90 receptors, CD117 receptors, HLA-DR, VEGFR-1, VEGFR-2, Muc-18 (CD146), CD130, stem cell antigen (Sca-1), stem cell factor 1 (SCF/c-Kit ligand), Tie-2 and HAD-DR.

87. The medical device of claim 84 wherein the first layer further comprises a one or more of the following cytokines: beta interferon, gamma interferon, and tumor necrosis factor.
